(19) United States
(12) Patent Application Publication
(10) Pub. No.: US 2005/0220875 A1
(43) Pub. Date: Oct. 6, 2005

(54) Sustained-release tablet formulation

(75) Inventors: Yi Yan Yang, Singapore (SG); Cherng-wen, Darren Tan, Singapore (SG); Shabbir M. Moochhala, Singapore (SG); Lishan Wang, Singapore (SG); Donna Tan, Singapore (SG)

Correspondence Address: KLARQUIST SPARKMAN, LLP, 121 SW SALMON STREET, SUITE 1600, PORTLAND, OR, 97204, US

(73) Assignees: Agency for Science, Technology and Research; DSO National Laboratories

(21) Appl. No.: 10/815,987

(22) Filed: Mar. 31, 2004

(51) Int. Cl. A61K 31/522; A61K 9/48; A61K 9/22

(52) U.S. Cl. 424/468; 514/263.31; 424/454; 514/454

(57) ABSTRACT

The invention provides a sustained-release tablet that can release caffeine and other xanthine derived stimulants at a nearly constant rate. The tablet comprises a hydrophilic polymer of high molecular weight and in one embodiment, the tablet includes caffeine and poly(ethylene oxide) of molecular weight of about 4×10⁶ to 8×10⁶. Sustained delivery of caffeine and other xanthine-derived stimulants is possible with a low concentration of the polymer and moreover, a wide range of concentration of caffeine and other stimulants can be released at a nearly constant rate.

### FIELD OF THE INVENTION

The invention relates to a sustained-release tablet formulation, including a sustained-release caffeine tablet.

### BACKGROUND OF THE INVENTION

Caffeine is typically used for its psychomotor stimulant action. For example, caffeine may be ingested to maintain alertness for working night shifts, for late-night studying, or during military operations. The stimulating effect of this compound depends upon the plasma level in the user's body. Caffeine is rapidly absorbed by the oral route, where its stimulating effect is rapid but transitory. The most common sources of caffeine are caffeine-containing beverages, such as coffee, tea, and certain carbonated beverages. However, one has to repeatedly consume these beverages in order to maintain systemic levels of caffeine. An unpleasant result of such consumption is the constant need to relieve oneself due to the ingestion of vast quantities of fluid. Furthermore, the caffeine levels in the plasma are difficult to predict with such consumption, which can result in jitteriness from over-consumption or lack of alertness from under-consumption.

Tablets containing caffeine are available commercially and include Cafergot, Anacin, Migril and Picapan. However, these tablets contain other pharmaceutically active agents other than caffeine, such as chlorpheniramine and ergotamine tartrate and contain only 15 to 30 mg of caffeine. Such low levels of caffeine can only maintain their presence in the blood for a few hours before the caffeine is metabolized thus requiring repeat dosing of the caffeine tablets every 3 to 4 hours to maintain the desired level of alertness.

A sustained release microparticulate caffeine formulation is disclosed in U.S. Pat. No. 5,700,484. Each microparticle is in the form of a solid core with a layer of biodegradable matrix containing caffeine surrounding the core and additional layers may be included. The method of preparing this formulation is complicated.

### SUMMARY OF THE INVENTION

In one aspect, the present invention provides a sustained-release tablet comprising caffeine and a hydrophilic polymer. Caffeine is released from the tablet at a nearly constant rate. The tablet may also comprise other xanthine-derived stimulants, instead of or in addition to caffeine. In another aspect therefore, the invention provides a sustained-release tablet comprising at least about 40% xanthine-derived stimulant by weight of the tablet and a hydrophilic polymer.

In a further aspect, the invention provides a method for increasing the alertness of a subject comprising orally administering a tablet according to the invention. A method of preparing a sustained-release tablet is also provided comprising the steps of mixing caffeine or other xanthine-derived stimulant with a hydrophilic polymer to form a mixture and compressing the mixture into a tablet.

### BRIEF DESCRIPTION OF THE FIGURES

FIG. 1 is a graph showing the release profiles of tablets containing various concentrations of caffeine, formulated using poly(ethylene oxide) (PEO) of molecular weight 4×10⁶.

FIG. 2 is a graph showing the release profiles of tablets containing various concentrations of caffeine, formulated using PEO of molecular weight 8×10⁶.

FIG. 3 is diagram of a donut shaped caffeine tablet according to one embodiment of the invention.

FIG. 4 is a graph showing the release profiles of the donut shaped tablet containing 80% caffeine formulated using PEO of molecular weight 8×10⁶.

FIG. 5 is a graph showing the release profiles of the donut shaped tablet containing 33.3% caffeine formulated using PEO of molecular weight 8×10⁶.

FIG. 6 is a graph showing the release profiles in vivo of caffeine tablets formulated according to an embodiment of the invention. The release profiles of control tablets formulated with sucrose are also shown.

### DETAILED DESCRIPTION OF THE INVENTION

The invention in one embodiment provides a sustained-release caffeine tablet comprising caffeine and a hydrophilic polymer. The term sustained-release tablet describes a tablet that achieves a slower or prolonged release of drug over a period of time when compared to a conventional tablet.

In an illustrative embodiment of the invention, the polymer is poly(ethylene oxide)(PEO) having a molecular weight of about 4×10⁶ or greater. PEO of a wide range of molecular weights is available under the trade name Polyox. In one embodiment, the MW of PEO is in the range of about 4×10⁶ to 8×10⁶. PEO of molecular weight greater than 8×10⁶ may also be used. However, where desirable, the molecular weight selected should not interfere with the nearly constant rate of release of caffeine discussed below.

PEO that are commercially available can include PEO having a range of molecular weights. For example, PEO of molecular weight in the range of about 4×10⁶ to 8×10⁶ may comprise PEO of molecular weights ranging from about 600 to 8×10⁶. Additionally, a small amount of PEO of a lower molecular weight may be added to PEO without affecting the release characteristics of caffeine and as will be well understood in the art, reference to any particular molecular weight therefore is not intended to preclude the presence or small addition of PEO of lower molecular weight. In different embodiments, low molecular weight polyethylene glycol (PEG) for example, less than about 10,000 (Mn) may also be included provided it is present in an amount that does not affect the release characteristics of caffeine. The terms molecular weight and MW are used interchangeably herein and unless otherwise specified, refer to weight average molecular weight.

Other hydrophillic polymers of varying molecular weight and viscosity may be used. In one embodiment, the polymer may be hydroxypropylmethylcellulose (HPMC). In one embodiment, HPMC has a viscosity of at least about 40 centipoise (as measured in a 2% aqueous solution at 20° C.). In other embodiments, the viscosity is about 40 to 100,000 centipoise. Such HPMC may be obtained from, for example, Sigma Chemical Company, St. Louis, USA.

In other embodiments, the polymer may be cellulose acetate (e.g. CA-398-10NF, molecular weight of about 40,000 Mn) from Eastman), cellulose acetate butyrate (e.g. CAB-381-2 molecular weight of about 40,000 Nn), CAB-500-5-Mn 57,000 from Eastman), polyvinylpyrrolidone (PVP) (e.g. from Sigma Chemical Company, St. Louis, USA), or sodium carboxymethyl cellulose (SCMC) (e.g. from Sigma Chemical Company, St. Louis, USA). In different embodiments, PVP has a molecular weight of about 1×10⁶ (Mn) or greater and SCMC has a molecular weight of about 3,000 (Mn) or greater.

Caffeine (1,3,7 trimethyl xanthine) which has the structure shown below, is a xanthine derivative and a stimulant, widely used to increase alertness. Caffeine has a molecular weight of 194.2 and a water solubility of 20 mg/mL:

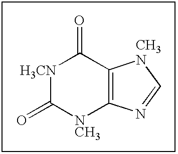

In various embodiments, the caffeine concentration in the tablet, by weight, is about 8 to 90% and the PEO concentration, by weight, is about 92 to 10%. At such concentrations, caffeine can be delivered at a nearly constant rate over a period of about 8 to 24 hours after oral administration. The term “nearly constant rate” is intended to describe a rate of release which is approximately linear, or approximately zero-order as shown in the Figures and further described in the Examples for various embodiments of this invention. The exponent for the release kinetics (n) reflects the linearity of the caffeine release. In some embodiments, n is greater than about 0.60. In other embodiments, n is greater that about 0.70 and greater that about 0.90.

This would permit the maintenance of a constant systemic concentration of caffeine, after oral administration for a period of about 8 to 24 hours, thereby requiring administration of only one to three doses in a 24-hour period.

Since the rate of caffeine release does not significantly vary as a function of the caffeine concentration, tablets can be prepared with a wide range of caffeine concentrations, while maintaining a nearly constant rate of release.

Since caffeine is a water-soluble drug, it might be expected that a sustained-release formulation would require a high proportion of polymer in order to effect sufficient retardation of caffeine release. It was therefore surprising that at PEO concentration as low as about 10 to 20% by weight, a sustained delivery of caffeine can be achieved. In various embodiments, the caffeine concentration is about 50% by weight of the tablet and about 80 to 90% by weight of the tablet. In one embodiment, the caffeine concentration is about 90% by weight of the tablet. Moreover, the use of a higher MW polymer avoids the necessity of using a higher proportion of polymer per tablets, providing a considerable saving in the cost of the final product.

The level of caffeine required to maintain the stimulating effect of caffeine in a subject may vary depending on various factors such as the amount of caffeinated foods or beverages consumed by the subject, the weight and metabolic rate of the subject, etc. While a wide range of caffeine concentrations therefore may be suitable, it is expected that tablets which include about 100 to 700 mg caffeine can maintain sufficient plasma levels of caffeine to maintain alertness in a subject weighing about 70 kg for a period of about 8 to 24 hours. In one embodiment, one to three 360 mg tablets containing 80% caffeine in weight may be suitable. In some cases, it may be desirable to include a relaxant to minimize side effects associated with caffeine, such as restlessness and nervousness. In one embodiment, the relaxant may be a kavalactone, or kava, which is described in U.S. Pat. No. 5,977,120 and which is known to induce general relaxation in humans when orally ingested. In various embodiments, the tablets comprise about 2% to 50% by weight kavalactones.

The tablets consisting only of caffeine and a high molecular weight hydrophilic polymer (such as PEO having an average molecular weight in the range of about 4×10⁶ to 8×10⁶) can release caffeine at a nearly constant rate, without the need for additives such as inorganic salts and other solubilizers and lubricants which have in the past been added to achieve zero-order release kinetics.

In one embodiment therefore, the tablet consists of caffeine and PEO. The lack of any additional components reduces the risks of side-effects associated with these additional components. The lack of additional components also results in costs savings, both in terms of costs of materials, as well as costs associated with manufacture of the tablets.

In another embodiment, the tablet may consist essentially of caffeine and a high molecular weight hydrophilic polymer, for example PEO having an average molecular weight of 4×10⁶ or greater. Such tablets, while excluding other active ingredients and polymers, do not exclude additives, excipients or diluents that may be added without affecting the caffeine release characteristics (ie. without affecting a nearly constant rate of release), for example a lubricant to facilitate high speed manufacture of tablets. Lubricants are generally used at concentrations less than about 1% of the total weight of the tablet.

In a further embodiment, the tablets may be provided with a hole to improve the linearity of the caffeine release. By providing a hole, the surface area of the tablet remains nearly constant as the tablet erodes, maintaining nearly constant release rates of caffeine as the tablet erodes. In one embodiment, the hole may be provided in the middle to form a donut-shaped tablet.

The tablets in accordance with the present invention may be prepared by mixing caffeine and the polymer to form a mixture and compressing the resulting mixture into tablets by conventional means. The tablet in one embodiment is a homogenous mixture. In various embodiments, other additives described above may be mixed with caffeine and PEO prior to compression.

The tablets may be used for improving cognitive functions by enhancing alertness and increasing cerebral blood flow. In one aspect, the tablets may be administered orally to increase the alertness of a subject and in one aspect, the invention relates to a method of increasing the alertness of a subject comprising orally administering a tablet according to the invention. The desired caffeine concentration and the duration of caffeine release will vary depending on the need of the subject. In various embodiments, the tablet consists of about 8 to 90% caffeine by weight and poly(ethylene oxide) having an average molecular weight in the range of about 4×10⁶ to 8×10⁶, and caffeine is released at a nearly constant rate over a period of about 8 to 24 hours after oral administration.

While reference is made to caffeine herein, caffeine is just one example of a xanthine derivative which is a stimulant. Others include aminophylline, pentoxifylline, oxtriphylline, theobromine and theophylline and other similar xanthine or xanthine-derivative stimulants (termed herein xanthine-derived stimulant), may be used instead of, or in addition to caffeine as described above. Aminophylline, theobromine and theophylline can be purchased from, for example, Sigma Chemical Company, St. Louis, USA. Oxtriphylline can be purchased from, for example, Maple Leaf Meds., Canada.

In one embodiment, the tablet comprises at least about 40% xanthine-derived stimulant by weight of the tablet (which may be a mixture of xanthine-derived stimulants) and a high molecular weight hydrophilic polymer, as described above. In one embodiment, the tablet may consist of the stimulant and PEO and the tablet may comprise about 50% or about 80 to 90% of the stimulant, by weight of the tablet.

The structure of other xanthine-derived stimulants are shown below.

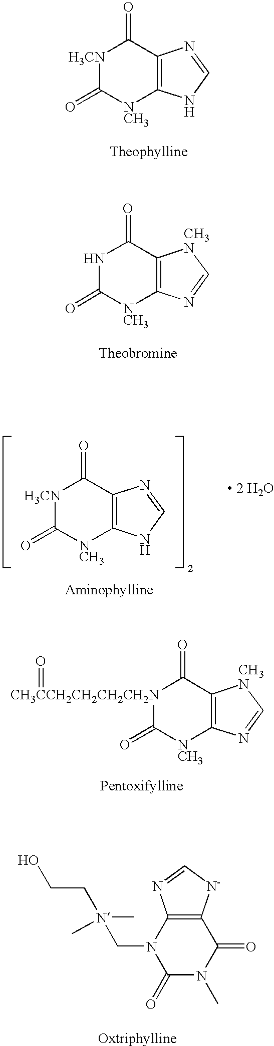

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way.

The word “comprising” is used as an open-ended term, substantially equivalent to the phrase “including, but not limited to”. The following examples are illustrative of various aspects of the invention, and do not limit the broad aspects of the invention as disclosed herein.

All documents referred to herein are fully incorporated by reference.

### EXAMPLES

### Example 1

PEO of molecular weight of 4×10⁶ and 8×10⁶ was obtained from Aldrich Chemical Company Inc., Milwaukee, USA (Cat. No. 37 283-8). Caffeine U.S.P. was obtained from Sigma Chemical Company, St. Louis, USA (Cat. No. C-8960).

Appropriate quantities of caffeine and PEO were mixed thoroughly by grinding. No other excipients were added. The resultant mixture was then compressed with a laboratory hydraulic press (Graseby Specac) under a pressure of 38 MPa for 1 minute using two 10 mm-diameter tablet punches with convex surfaces. The total mass of each 10 mm-diameter biconvex tablet was 360 mg.

Simulated intestinal fluid, hereafter abbreviated to SIF [0.68 % (w/v) K₂HPO₄+19% (v/v) 0.2 N NaOH, pH 7.5±0.1] without pancreatin was prepared as described by the United States Pharmacopeia XXIII. The procedure was as follows: dissolve 6.8 g of monobasic potassium phosphate in 250 mL of water, mix, and add 190 mL of 0.2 N sodium hydroxide and 400 mL of water. Adjust the resulting solution with 0.2 N sodium hydroxide to a pH of 7.5±0.1. Dilute with water to 1000 mL.

The in vitro release profile of each tablet was determined using a VK 7000 dissolution testing station (VanKel Technology Group, Weston Parkway, USA), with 1000 mL sample vessels. Each vessel was filled with 900 mL of SIF as the receptor medium. Aliquots of 1 mL were removed from each vessel at predetermined time intervals. Each tablet was studied in triplicate.

Each aliquot was filtered through a 0.22 μm filter and analysed using a Waters HPLC system consisting of a 2690 separations module (Waters, USA) and 996 photodiode array detector (Waters, USA). The UV detector wavelength was set at 270 nm. Separation was achieved using a SymmetryShieldTM RP8 analytical column (4.6×150 mm, 5 μm) and a Sentry TM SymmetryShieldTM RP8 guard column (3.9×20 mm, 5 μm) (Waters, USA) at room temperature. A mobile phase of 70% deionised water and 30% methanol, at a flow rate of 1 mL min⁻¹ was used. The injection volume used was 10 μl.

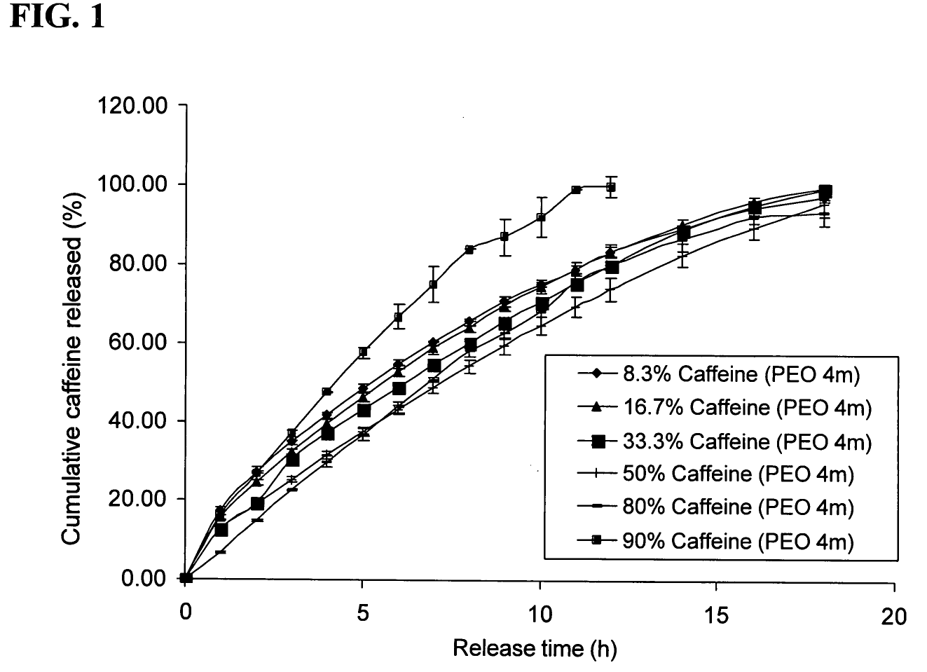

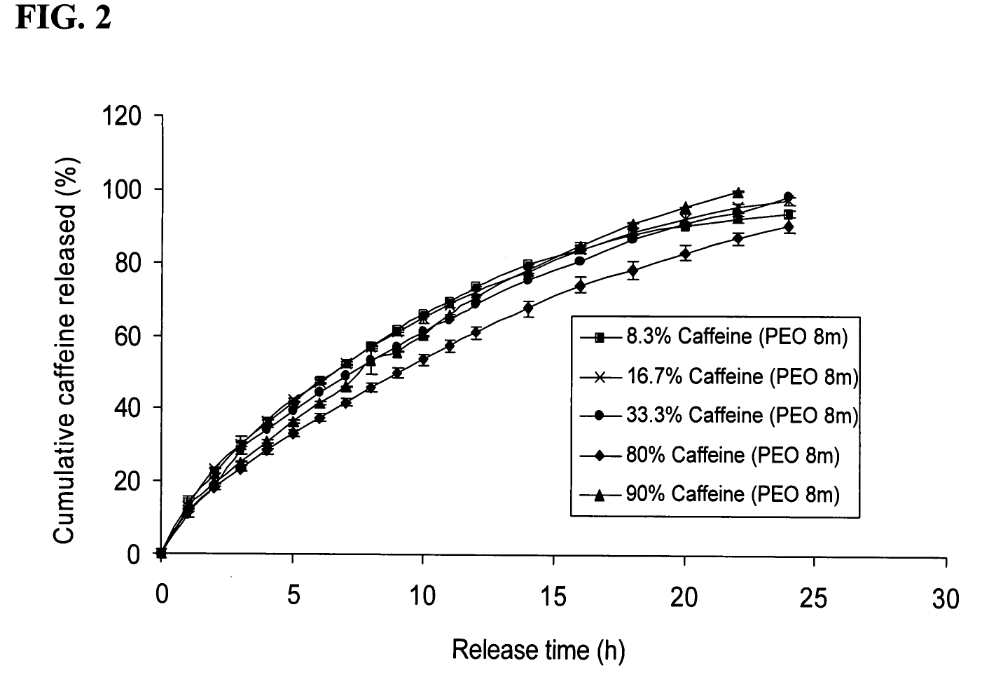

FIGS. 1 and 2 show the caffeine release profiles of tablets containing PEO of molecular weight 4×10⁶ and 8×10⁶, respectively. Specifically, the graphs plot the cumulative percentage of caffeine released against time, measured in hours. Both types of tablets were made using six different caffeine concentrations w/w: 8.3%, 16.7%, 33.3%, 50%, 80% and 90%.

As shown in FIGS. 1 and 2, the tablets comprising PEO of MW=4×10⁶ and 8×10⁶, having caffeine concentrations ranging from 8.3% to 90%, displayed nearly zero-order kinetics, resulting in sustained release of caffeine from the tablets over 11, 18, 22 and 24 hours, as described more fully below.

FIGS. 1 and 2 show that tablets comprising the higher molecular weight PEO (8×10⁶) released their caffeine at a lower rate than the tablets with PEO of MW=4×10⁶. This is expected since the erosion, and hence release of caffeine, of PEO (8×10⁶) would be slower than that of PEO (4×10⁶. Thus, for example, as shown in FIG. 1, tablets with PEO having a molecular weight of 4×10⁶ achieved a sustained release of caffeine over 11 and 18 hours at concentration levels of 90% and 8.3-80%, respectively. However, as shown in FIG. 2, tablets with PEO having a molecular weight of 8×10⁶ yielded sustained release of caffeine over a more extended period of 22 and 24 hours at 90% and 8.3-80% caffeine concentrations, respectively.

The release profiles shown in FIGS. 1 and 2 also illustrate that the tablets having PEO of MW=4×10⁶ or 8×10⁶ display similar release profiles over a wide range of caffeine concentrations for each MW of PEO, specifically, for caffeine concentrations of 8.3% to 80%.

As shown in FIGS. 1 and 2, caffeine concentrations of up to 90% did not affect the release profile of tablets having PEO of molecular weights 8×10⁶. However, when the caffeine concentration was 90%, the release of caffeine was faster for tablets made with PEO of 4×10⁶

The ability to use high concentrations of caffeine in the formulation without affecting the release profile of the formulation, results in decreased costs associated with their manufacture as there is a resultant decrease in proportion of polymer necessary.

The release kinetic data were determined by the following phenomenological equation: 1 nMₜ/M_{∝}=1nk+n1nt, where Mₜ, M_{∝}, k and n are the amounts of caffeine released at time t, the total amount of caffeine in the tablet, the constant, and exponent for the release kinetics, respectively. A linear regression analysis was performed and a correlation coefficient R² was obtained. The value of n displayed in the figures reflects the linearity of the caffeine release profile. The closer to 1 the value of n is, the more linear the release profile. The n and R² values for FIGS. 1 and 2 are shown below.

TABLE 1

| | PEO (MW = 4,000,000) | | PEO (MW = 8,000,000) | |
|---|---|---|---|---|
| | (FIG. 1) | | (FIG. 2) | |
| | | Correlation | | Correlation |
| | | coefficient | | coefficient |
| Drug conc | Exponent n | R² | Exponent N | R² |
| 8.3% | 0.6358 | 0.9999 | 0.6803 | 0.9995 |
| 16.7% | 0.6783 | 0.9995 | 0.6910 | 0.9967 |
| 33.3% | 0.7635 | 0.9923 | 0.7437 | 0.9924 |
| 50% | 0.7127 | 0.9957 | 0.6624 | 0.9994 |
| 80% | 1.0275 | 0.9991 | 0.6617 | 0.9981 |
| 90% | 0.7539 | 0.9999 | 0.6697 | 0.9962 |

### Example 2

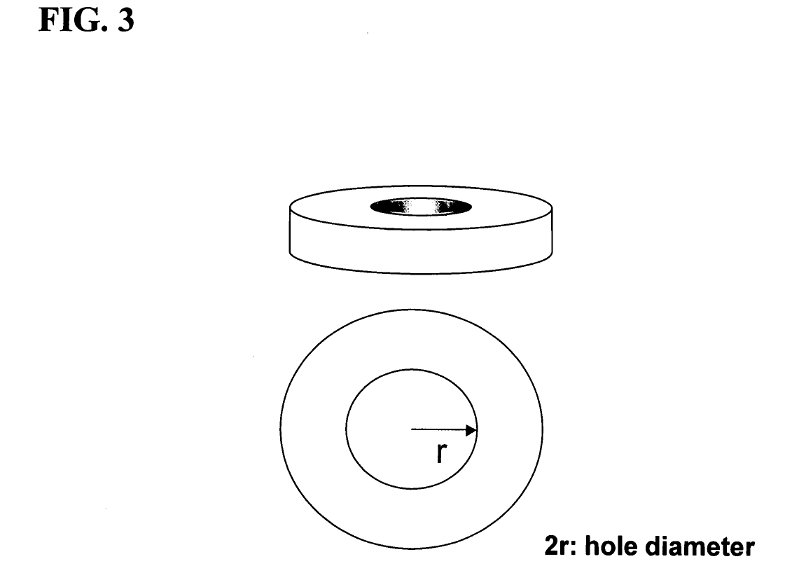

A donut-shaped tablet, an example of which is shown in FIG. 3, was prepared to improve linearity of the caffeine release profiles. Each tablet was formulated as described in Example 1, but was compressed using a 10 mm-diameter tablet punch having a central steel cylinder of varying diameter, as shown below. The central cylinder was used to form the holes in the middle of the tablets.

[image]

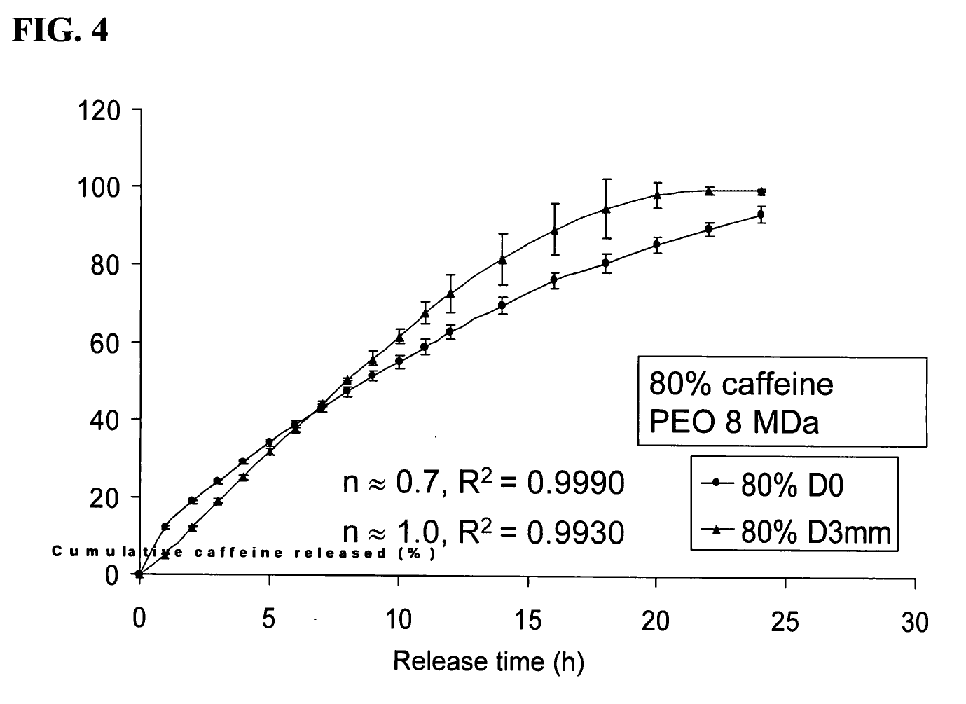

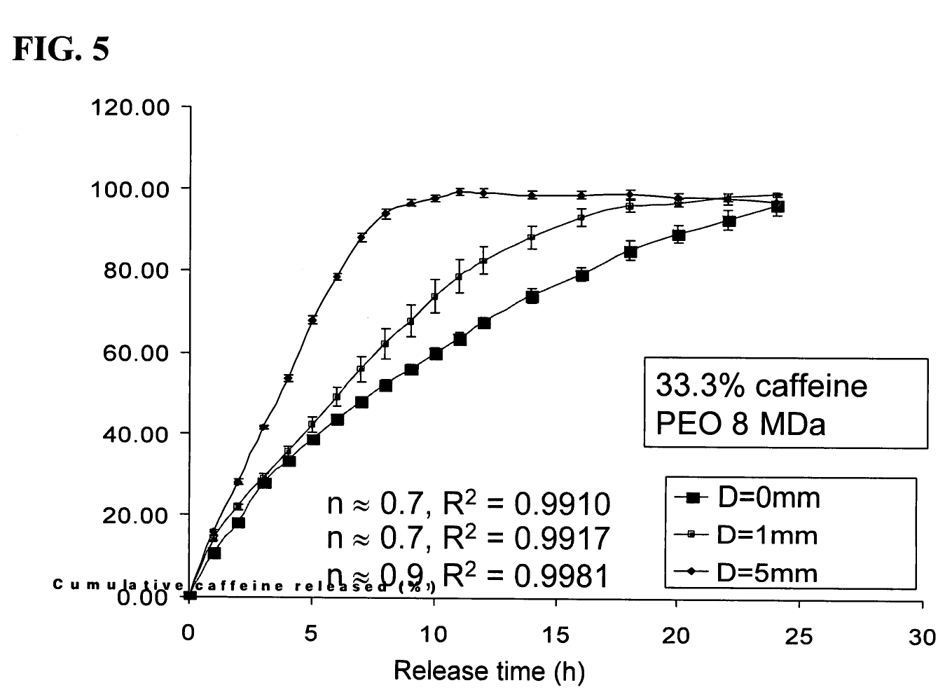

FIGS. 4 and 5 display the caffeine release profiles of tablets containing PEO of average molecular weight 8×10⁶ with a hole of different diameters with caffeine concentrations of 80% and 33.3%, respectively. The n values were calculated according to the equation provided in paragraph 46, above.

As shown in FIGS. 4 and 5, the linearity of caffeine release was improved by using donut-shaped tablets. This is due to the fact that these tablets kept relatively constant surface area during the process of erosion. As the surface area of the conventional tablets (ie. with no hole or D=0) decreases with the progression of erosion, the caffeine release rate decreases over time.

Increasing the surface area of the tablets increased the rate of caffeine release. For example, as shown in FIG. 5, at a caffeine concentration of 33.3%, about 94% caffeine was released within 8, 16 and 24 hours from the tablets with a hole of 5, 1 and 0 mm, respectively. As shown in FIG. 4, at a caffeine concentration of 80%, about 94% caffeine was released within 18 and 24 hours from the tablets with a hole of 3 mm and without a hole, respectively.

### Example 3

For the in vivo experiments, the weighed PEO and caffeine were mixed thoroughly by manually grinding in a stone mortar. Separately, sucrose was used in the matrix, in place of PEO, to serve as a negative control. The resultant powder mixture was then compressed with a laboratory hydraulic press (Graseby Specac) under a pressure of 38 MPa for 1 minute using two 5 mm-diameter tablet punches with convex surfaces. The total mass of each 5 mm-diameter curved tablet was kept at 35 mg.

The experiments were performed on Sprague-Dawley rats weighing 280-300 g (Laboratory Animals Centre, Sembawang, Singapore). The rats were first anaesthetised with a hypnorm/dormicum mixture administered intra-peritoneal, at 0.33 mL/100 g rat.

The left femoral vein was then located and isolated by making a small, shallow incision at the intersection of an imaginary mid-femoral line with an imaginary line from the hip to the base of the tail. The underlying fat and connective tissue was teased away from the blood vessels using a pair of blunt Mayo, straight scissors. The vein was cleared of adhering fat and connective tissue, and supported using a small rectangular piece of cardboard.

Two short strands of suture were then passed beneath the vein, and moved to the proximal and distal ends of the exposed vein. The distal strand was stitched into a dead knot to restrict venous flow, while the proximal end was tied into a loose knot to allow entry of the catheter later.

A small incision was made near the distal end of the vein, using a pair of Vannas micro-scissors. A heparinised catheter was then inserted elastin-end first into the vein toward the heart, with the help of a pair of curved forceps. Insertion was complete once the PE10 section was completely in the vein.

Proper vascular access was then determined by checking for venous back flow using a heparinised syringe. The entire length of the cannula was flushed with heparinised-saline solution. The catheter was then plugged with a small stretch of metal.

The catheter was then secured using the suture at the distal end of the vein, by making a dead knot about the catheter. The proximal end of the vein was in turn secured with a dead knot, making sure not to occlude the catheter with a knot that was too tight.

A suture was then passed through the fat tissue lying on one side of the catheter, passed under the vein, and once again, through the muscular tissue lying on the other side of the vein. This suture was left in place. The rectangular piece of cardboard was removed.

The rat was then turned over to expose the dorsal surface. A small incision was made on the skin between the ears without damaging the underlying connective tissue. A steel rod was then introduced under the skin and pushed toward the area of the cannulated femoral vein. It was made to emerge close to the site of cannulation.

A polymeric tube was then inserted using the steel rod as a trocar, and the rod was removed, leaving the plastic access-way in place. The distal end of the catheter was then passed through the tubing and made to emerge from between the ears of the rat.

The catheter was finally secured in place using sutures. Again, care was taken out to occlude the catheter. Finally, an epoxy mix was prepared and poured onto the catheter to cement it to the skin.

The rats were then allowed to recuperate for at least 24 hours before the experiments were commenced.

Tablets (PEO/caffeine and sucrose/caffeine) and caffeine were force-fed into the rats by clasping each tablet with the tips of a forceps and pushing the tablet into the posterior of the rat pharynx. The tablets were held in place for a while to induce the rat to swallow the tablet. Caffeine dissolved in deionised water at volume concentrations corresponding to the mass concentrations of the tablets were dispensed using a rigid dosing gavage needle directly into the stomach.

Immediately before administration of the tablets and solutions, a 500 μL sample of blood was taken at t=0 hrs (pre-dose), through the exposed catheter. Subsequent samples were drawn from the catheter at 1, 2, 3, 4, 5, 6, 7, 8, 10, 12, 24, 30, 36 and 48 hours after drug administration. After each withdrawal, an equal volume of 0.9% normal saline was injected back into the blood stream to minimize loss of body fluid. Water and food were available ad libitum in the metabolic cages.

Each 500 μL volume of blood was collected in heparinised microcentrifuge tubes and centrifuged under 200 g for 5 minutes to obtain the plasma. All plasma samples were stored at −70° C. in fresh heparinised microcentrifuge tubes until the caffeine could be extracted from it prior to analysis by high performance liquid chromatography (HPLC).

To extract the caffeine from the plasma samples, 150 μL of plasma from each sample, 150 μL of an internal standard (I.S., 500 μg mL⁻¹ N-acetyl-p-aminophenol in water), and an additional 3 mL of dichloromethane-isopropanol (88:12, v/v) were placed in a 120 mm glass tube, vortex-mixed and placed on a shaker for 45 min. After centrifugation for 10 min at 11 g, the organic phase was transferred into a clean glass tube and evaporated to dryness under a gentle stream of nitrogen. The extract was reconstituted with 200 μL of 0.05% (v/v) acetic acid-methanol solution (92:8).

Each reconstituted sample of caffeine was analysed for caffeine quantity using a chromatographic HPLC system (Waters 2690 Separation Module) consisting of a 600E multi-solvent delivery system pump, Ultra WISP 715 auto-injector and a UV-Vis 996 photodiode array detector, all obtained from Waters Asia Ltd. An injection volume of 50 μL was used. The detector was operated at a wavelength of 254 nm. The caffeine metabolites were separated on a SymmetryShield™ Cartridge Column RP18 (250×4.6 mm I.D.; particle size, 5 μm) from Waters Asia Ltd at 22° C. and a flow rate of 1 mL min⁻¹. A mobile phase comprising 0.05% acetic acid, 50 mM ammonium acetate and methanol was used. The mobile phase consisting of 0.05% acetic acid, 50 mM ammonium acetate and methanol was programmed according to the following gradient schedule:

TABLE 2
Gradient Program for HPLC Analysis of Reconstituted Caffeine

| Time | Flowrate | | | |
|---|---|---|---|---|
| (min) | (ml/min) | Solvent A (%) | Solvent B (%) | Solvent C (%) |
| 0 | 1.00 | 100 | 0 | 0 |
| 5 | 1.00 | 95 | 0 | 5 |
| 22 | 1.00 | 95 | 0 | 5 |
| 24 | 1.00 | 0 | 95 | 5 |
| 35 | 1.00 | 0 | 95 | 5 |
| 45 | 1.00 | 0 | 60 | 40 |
| 50 | 1.00 | 0 | 60 | 40 |
| 51 | 1.00 | 100 | 0 | 0 |
| 56 | 1.00 | 100 | 0 | 0 |

Solvent A: 0.05% acetic acid
Solvent B: 50 mM ammonium acetate
Solvent C: methanol

Sustained delivery of caffeine was expected to result in two phenomena: a reduction in the initial high rate of caffeine release (burst release), as well as a reduction in the change in caffeine concentration in the systemic circulation.

In vitro studies showed that the release profile of the caffeine-loaded PEO tablets was not affected by the quantity of caffeine used, at least up to about 80% by weight. Therefore, 32% (w/w) caffeine, with matrices of PEO (MW=8×10⁶) were used.

Tablets comprising sucrose as the matrix and either 8% or 32% (w/w) caffeine served as the negative control. In a rat, the 8% caffeine loading is the approximate equivalent of a single conventional dose of caffeine (e.g. in beverages). Thus, the 32% caffeine load in the PEO tablets in the rat provides the equivalent amount of caffeine that would result from four conventional doses, or one high dose, of caffeine. This simulates the scenario where a person consumes four cups of a caffeine-containing beverage in order to attain a specific systemic caffeine concentration.

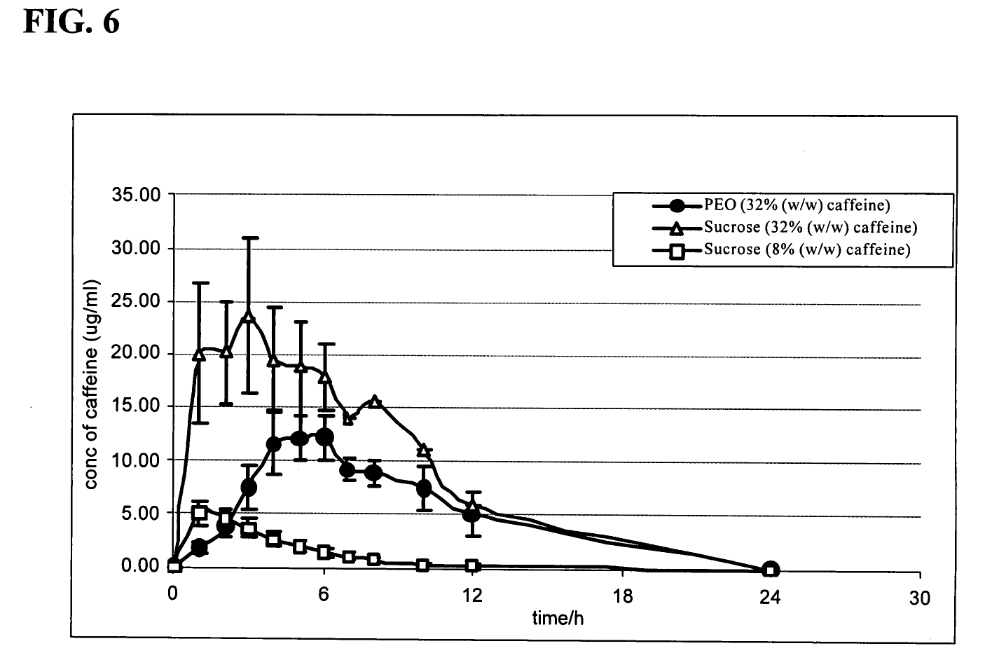

FIG. 6 shows that, in the case of the 32% caffeine-containing sucrose tablets, there was a faster initial increase in serum caffeine concentration due to the almost immediate release of caffeine from the tablets, and the subsequent absorption of the caffeine by the gastro-intestinal tract. In contrast, caffeine concentrations in the serum of the rat fed with 32% PEO tablets did not increase as quickly as the sucrose tablets, despite the rats having similar absorption rates, suggesting that the PEO retarded the release of caffeine from the tablets.

The sucrose control tablets containing 32% caffeine appeared to have a somewhat similar release profile as the PEO tablets. This is likely because the caffeine could not be metabolized as quickly as it was being absorbed into the serum due to enzyme saturation. Thus, with respect to the sucrose tablets, what appears in FIG. 6 to be sustained release of caffeine is probably a sustained presence of caffeine. However, in the case of the PEO tablets, as shown in FIG. 6, the persistence of high caffeine concentration in the serum is due to a constant supply of caffeine (as it is slowly released from the sustained-release tablet) and subsequent absorption into the serum. In the case of the 32% PEO tablet, the plateau does not indicate an accumulation of caffeine in the plasma, since the plasma caffeine level is lower than that seen in the 32% sucrose tablet. Instead, this plateau should be interpreted as the constant replenishment of plasma caffeine levels upon their removal by metabolic enzymes.

These results can be interpreted to mean that a 32% (w/w) caffeine PEO tablet was able to maintain a systemic caffeine concentration that was only possible using 2 to 4 of the 8% (w/w) caffeine sucrose tablet. That is to say, since the 8% sucrose tablets are meant to represent a conventional dose of caffeine in, for example, a caffeine beverage, the results indicate that a 32% PEO tablet can achieve the equivalent of about 4 doses of such a beverage.

A simple two-component system for a sustained-release caffeine formulation therefore has been achieved.

1. A sustained-release tablet comprising caffeine and a hydrophilic polymer wherein caffeine is released from the tablet at a nearly constant rate.

2. The tablet according to claim 1 wherein the polymer is hydroxypropylmethylcellulose (HPMC), cellulose acetate, cellulose acetate butyrate, polyvinylpyrrolidone or sodium carboxymethyl cellulose.

3. The tablet according to claim 2 which is a homogeneous mixture.

4. The tablet according to claim 3 comprising about 8% to 90% caffeine by weight of tablet.

5. The tablet according to claim 1 wherein the polymer is poly(ethylene oxide) having a molecular weight of about 4×10⁶ or greater.

6. The tablet according to claim 5 which is a homogeneous mixture.

7. The tablet according to claim 6 comprising about 8% to 90% caffeine by weight of tablet.

8. The tablet according to claim 7 wherein poly(ethylene oxide) has a molecular weight in the range of about 4×10⁶ to 8×10⁶.

9. The tablet according to claim 8 comprising about 10 to 92% poly(ethylene oxide) by weight of tablet.

10. The tablet according to claim 9 wherein caffeine is released over a period of about 8 to 24 hours after oral administration.

11. The tablet according to claim 10 comprising a kavalactone.

12. The tablet according to claim 10 wherein the tablet is donut-shaped.

13. The tablet according to claim 7 consisting of poly(ethylene oxide) and caffeine.

14. The tablet according to claim 7 consisting essentially of poly(ethylene oxide) and caffeine.

15. The tablet according to claim 13 wherein poly(ethylene oxide) has a molecular weight in the range of about 4×10⁶ to 8×10⁶.

16. The tablet according to claim 15 comprising about 50% caffeine by weight of the tablet.

17. The tablet according to claim 15 comprising about 80 to 90% caffeine by weight of the tablet.

18. The tablet according to claim 15 wherein caffeine is released over a period of about 8 to 24 hours after oral administration.

19. The tablet according to claim 18 wherein the tablet is donut-shaped.

20. A sustained-release tablet comprising at least about 40% xanthine-derived stimulant by weight of the tablet and a hydrophilic polymer.

21. The tablet according to claim 20 wherein the stimulant is caffeine, aminophylline, oxtriphylline, theobromine, or theophylline or a mixture thereof.

22. The tablet according to claim 21 wherein the polymer is hydroxypropylmethylcellulose (HPMC), cellulose acetate, cellulose acetate butyrate, polyvinylpyrrolidone, or sodium carboxymethyl cellulose.

23. The tablet according to claim 21 wherein the polymer is poly(ethylene oxide) having a molecular weight of about 4×10⁶ or greater.

24. The tablet according to claim 23 wherein poly(ethylene oxide) has a molecular weight of about 4×10⁶ to 8×10⁶.

25. The tablet according to claim 23 which consists of the stimulant and poly(ethylene oxide).

26. The tablet according to claim 25 comprising about 50% of the stimulant by weight of the tablet.

27. The tablet according to claim 25 comprising about 80 to 90% of the stimulant by weight of the tablet.

28. A method of preparing a sustained-release caffeine tablet comprising,
mixing caffeine and a hydrophillic polymer to form a mixture; and
compressing the mixture into a tablet.

29. The method according to claim 28 wherein the polymer is poly(ethylene oxide) of a molecular weight of about 4×10⁶ or greater.

30. A method for increasing the alertness of a subject comprising orally administering a tablet comprising caffeine and poy(ethylene oxide) wherein poly(ethylene oxide) has a molecular weight of about 4×10⁶ or greater.

31. The method according to claim 30 wherein poly(ethylene oxide) has a molecular weight in the range of about 4×10⁶ to 8×10⁶.

32. The method according to claim 31 wherein the tablet comprises about 8% to 90% of caffeine by weight of the tablet.

33. The method according to claim 32 wherein the tablet consists of caffeine and poly(ethylene oxide).

34. The method according to claim 33 wherein caffeine is released at a nearly constant rate over a period of about 8 to 24 hours after oral administration.
